(19) United States
(12) Patent Application Publication
(10) Pub. No.: US 2018/0078902 A1
(43) Pub. Date: Mar. 22, 2018

(54) SYSTEMS, METHODS AND DEVICES FOR ELECTRO-OSMOTIC PROPULSION IN A MICROFLUIDIC ENVIRONMENT

(71) Applicant: CALIFORNIA INSTITUTE OF TECHNOLOGY, Pasadena, CA (US)

(72) Inventors: Daniel L. Magley, Weaverville, NC (US); Vinayak Narasimhan, Pasadena, CA (US); Hyuck Choo, San Marino, CA (US)

(21) Appl. No.: 15/710,774

(22) Filed: Sep. 20, 2017

Related U.S. Application Data
(60) Provisional application No. 62/397,447, filed on Sep. 21, 2016.

(51) Int. Cl. B01D 61/42; H01M 8/16; H01M 12/08; F03B 17/00; A61M 5/142; A61B 5/145

(52) U.S. Cl. CPC B01D 61/427; H01M 8/16; H01M 12/08; F05B 2210/11; A61M 5/14276; A61B 5/14532; F03B 17/00

(57) ABSTRACT

Systems, methods and devices are provided for electro-osmotic propulsion in a microfluidic environment. These systems, methods and devices can include a body having a channel comprising a pair of open ends, and a plurality of electrodes coupled to the body, wherein the electrodes are configured to generate a voltage and cause an electro-osmotic flow of a fluid through the channel. In many of the embodiments, an on-board power supply coupled to the body is provided for generating voltage across the electrodes. In some embodiments, the channel comprises a cylindrical shape having a circular cross-sectional area.

### CROSS-REFERENCE TO RELATED APPLICATIONS

This application claims priority to U.S. Provisional Application Ser. No. 62/397,447, filed Sep. 21, 2016, which is hereby expressly incorporated by reference in its entirety for all purposes.

### FIELD

The subject matter described herein relates to systems, methods, and devices for electro-osmotic propulsion in a microfluidic environment. In particular, provided herein are embodiments which include, for example, a device having a body that includes a channel and a plurality of electrodes coupled to the body, wherein the plurality of electrodes is configured to generate a voltage and cause an electro-osmotic flow of fluid through the channel.

### BACKGROUND

An efficient method of transporting structures and payloads in a microfluidic environment, such as the human circulatory system, holds much promise for a plethora of biomedical applications, including minimally-invasive surgery, targeted drug delivery, local measurement and diagnostics, biopsies, depositing of microimplants, microfluidic pumps, and in vivo gene therapies, to name a few. Yet, significant limitations exist with respect to the speed and power efficiency of known methods of transportation and propulsion. Previous studies, for example, have employed magnetic pulling or electro-osmotic helices to achieve speeds of only 20-30 body-lengths per second. Another study achieved a speed of 100 body-lengths per second, but used time-limited chemical reactions that lasted only 0.360 seconds.

Moreover, known devices and methods for propulsion in microfluidic environments have been too inefficient to allow for an on-board power supply and require external power sources. For example, one study from Stanford University utilized a magneto-hydrodynamic propulsion method and was able to achieve a speed of 1 body-length per second (0.53 cm/sec) using 250 μW. Current implantable bio-fuel cells, however, can provide approximately 218 uW/10 mm², which falls short of the 250 μW power requirement and leaves no power for additional functionality. As a result, this method and other known propulsion mechanisms in microfluidic environments require external power sources. In practice, this means that a patient with a device implanted into the circulatory system would potentially require bulky and expensive external equipment, such as large electromagnetic coils surrounding the patient's body, to power and navigate the device.

Accordingly, there is a present need for systems, methods, and/or devices that provide for a fast and power-efficient propulsion mechanism in a microfluidic environment, and which preferably allows for an on-board power supply.

### SUMMARY

Described herein are example embodiments of systems, methods, and devices for electro-osmotic propulsion in a microfluidic environment. These embodiments can operate by generating a voltage across a plurality of electrodes coupled to a structure having a channel with a pair of open ends, wherein the channel is disposed in the structure. The generated voltage can create an electro-osmotic flow of fluid through the channel. In some embodiments, an on-board power supply can be coupled to the structure to generate the desired voltage across the electrodes. Additionally, in some embodiments, the channel can comprise a cylindrical shape having a circular cross-sectional area through which the electro-osmotic flow of fluid occurs.

Other systems, devices, methods, features and advantages of the subject matter described herein will be or will become apparent to one with skill in the art upon examination of the following figures and detailed description. It is intended that all such additional systems, devices, methods, features and advantages be included within this description, be within the scope of the subject matter described herein, and be protected by the accompanying claims. In no way should the features of the example embodiments be construed as limiting the appended claims, absent express recitation of those features in the claims.

### BRIEF DESCRIPTION OF THE DRAWINGS

The details of the subject matter set forth herein, both as to its structure and operation, may be apparent by study of the accompanying figures, in which like reference numerals refer to like parts. The components in the figures are not necessarily to scale, emphasis instead being placed upon illustrating the principles of the subject matter. Moreover, all illustrations are intended to convey concepts, where relative sizes, shapes and other detailed attributes may be illustrated schematically rather than literally or precisely.

FIGS. 1A and 1B are diagrams illustrating electro-osmotic flow under different conditions.

FIG. 1C is a diagram illustrating a velocity profile associated with electro-osmotic flow in a cylinder.

FIG. 2A is a diagram from a computer simulation of electro-osmotic flow in an example embodiment.

FIG. 2B is a diagram depicting a draft profile for an example embodiment moving at various speeds in a fluid.

FIG. 2C is a graph showing the propulsive force of electro-osmosis flow as a function of channel diameter for an example embodiment.

FIGS. 3A and 3B are photographs showing an overhead view of electro-osmotic flow.

FIG. 4 is a perspective view of an example embodiment of a device for electro-osmotic propulsion in a microfluidic environment.

FIGS. 5A and 5B are graphs depicting, respectively, battery voltage and velocity as a function of time for an example embodiment of a device for electro-osmotic propulsion in a microfluidic environment.

FIG. 5C is a graphical plot depicting movement of one example embodiment of a device for electro-osmotic propulsion in a microfluidic environment.

FIGS. 6A to 6F are perspective views of example embodiments of devices for electro-osmotic propulsion in a microfluidic environment.

FIGS. 7A to 7E are side views of example embodiments of devices for electro-osmotic propulsion in a microfluidic environment.

FIG. 8 is a perspective view of an example embodiment of a structure including multiple devices for electro-osmotic propulsion in a microfluidic environment.

FIGS. 9A and 9B are block diagrams depicting an example embodiment of a biofuel cell for use with devices for electro-osmotic propulsion in a microfluidic environment.

### DETAILED DESCRIPTION

Before the present subject matter is described in detail, it is to be understood that this disclosure is not limited to the particular embodiments described, as such may, of course, vary. It is also to be understood that the terminology used herein is for the purpose of describing particular embodiments only, and is not intended to be limiting, since the scope of the present disclosure will be limited only by the appended claims.

As used herein and in the appended claims, the singular forms “a,” “an,” and “the” include plural referents unless the context clearly dictates otherwise.

The systems, methods, and devices described herein relate to electro-osmotic flow as a propulsion mechanism for a structure or device in a microfluidic environment. Accordingly, in many of the embodiments disclosed herein, a device comprising a body having a channel with a pair of open ends is provided. Two or more electrodes can be coupled to the body of the device, and can be configured to generate a voltage and cause an electro-osmotic flow of fluid through the channel. In some embodiments, the electrodes can be positioned on opposite sides of the body of the device, e.g., an anode can be coupled to one side of the body and a cathode can be coupled to another side of the body, opposite to the anode. Additionally, in some embodiments, the channel can comprise a cylindrical shape, having a circular cross-sectional area. However, other configurations and geometries for the body, channel and/or electrodes can be utilized with any of the embodiments described herein.

According to another aspect of the embodiments described herein, the device can also include an on-board power supply coupled to the body of the device. In some embodiments, the on-board power supply can be a biofuel cell or an aluminum-air battery. In other embodiments, however, the device can receive power from an external power supply that can be configured to emit a microwave or RF radiation.

Before describing more particular aspects of the embodiments in detail, however, it is first desirable to describe the principle of operation with respect to electro-osmosis as a propulsion mechanism, as well as examples of the accompanying structures, all of which can be used with the embodiments described herein.

### Electro-Osmosis as a Propulsion Mechanism

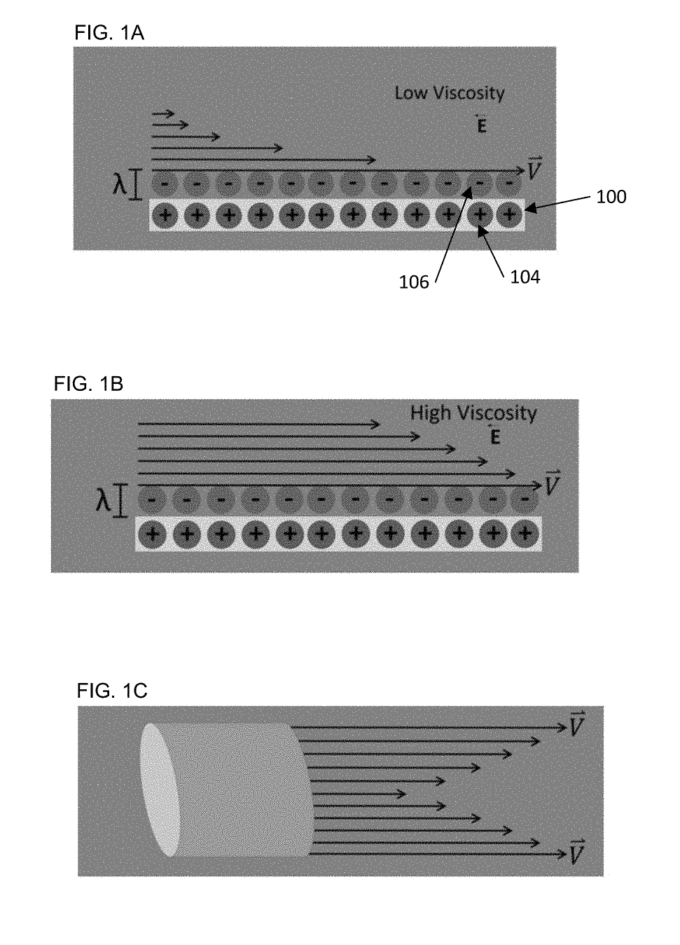

Electro-osmosis, or electro-osmotic flow, can be generally described as a phenomenon in which liquid flows through a capillary tube, porous material, membrane, microchannel, or other fluid conduit, in response to the application of an electric field. To illustrate, FIG. 1A is a diagram showing a solid structure 100 submerged in an ionic solution, where charged ions 104 have deposited on structure 100 to create a net charge on the surface. Those of skill in the art will appreciate that whether the deposited charges are positive or negative may vary depending on the material or the solution. The charged surface attracts oppositely charged particles 106. When an electric field is applied to the solution, a fluid layer, at a Debye length (λ) away from the structure, will move at a velocity that can be calculated using the Helmholtz-Smoluchowski equation, where [symbol] is velocity, ∈ is permittivity, δ is zeta potential (i.e., an intrinsic material property determined by the material and the solution type), is the viscosity of the fluid, and [symbol] is the electric field. Notably, the electric field is proportional to the velocity of the fluid.

$$\vec{V} = - \frac{ɛδ}{μ} \vec{E}$$

As illustrated in FIG. 1A, in a low viscosity environment (i.e., where there is a high Reynolds number), a dampening effect on the velocity of the fluid can be observed from layer to layer, such that velocity is diminished in those layers that are further away from the surface. By contrast, as illustrated in FIG. 1B, in a high viscosity environment (i.e., where there is a low Reynolds number), an efficient transfer of momentum occurs from layer to layer due to the viscosity of the fluid. As such, the dampening effect on velocity at distances further away from the surface is not as pronounced compared to low viscosity environments.

Utilizing these principles, an electro-osmotic propulsion mechanism employing a cylindrical channel can be implemented. As shown in FIG. 1C, a cylindrically shaped channel may be preferred because the boundary condition results in a net momentum transfer-based propulsion with the depicted velocity profile. In contrast to earlier propulsion mechanisms, such as those utilizing a helical tail, a cylindrical channel derives efficiency for layer-to-layer momentum transfer from viscosity and can more efficiently propel a structure. Furthermore, unlike with the helical tail, velocity vectors in a cylindrical channel can be substantially parallel to the intended direction of motion. As a result, less energy is wasted by ensuring that it all goes toward propulsion of a device.

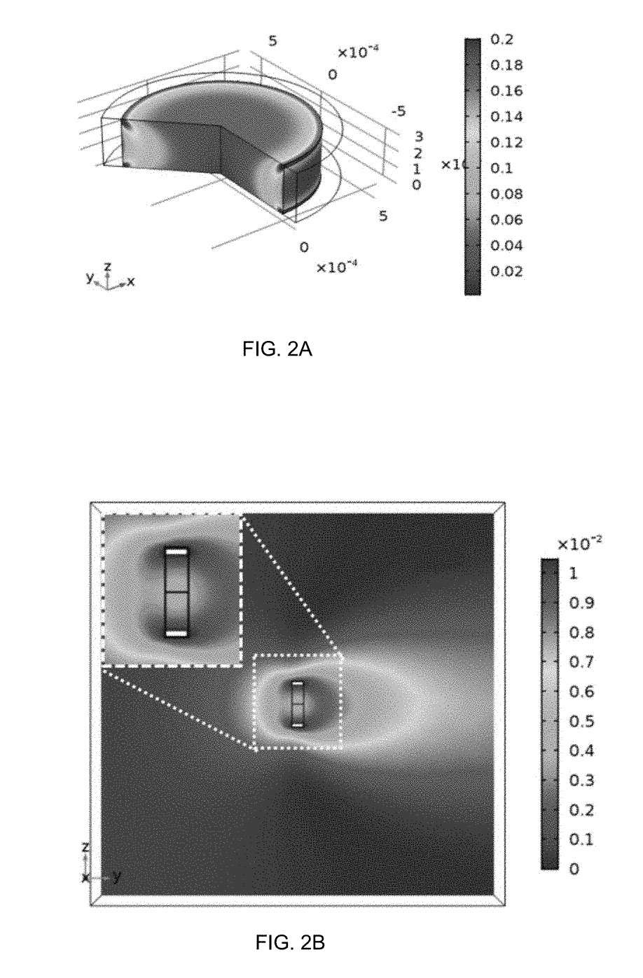

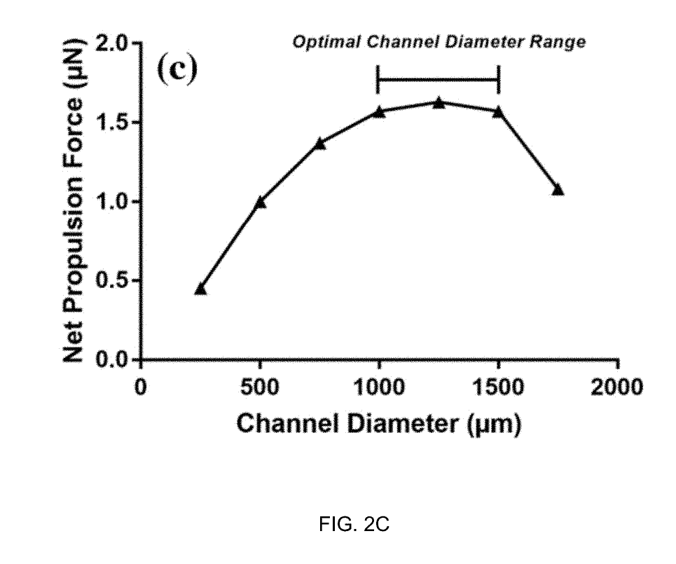

To demonstrate these principles and to determine optimal dimensions for the cylindrical channel having electrodes on opposite sides, computer simulations were performed using a COMSOL Microfluidics module, as shown in FIGS. 2A and 2B. Turning to FIG. 2A, a simulation of a fluid velocity profile inside a cylindrical channel when 250 mV is applied between electrodes is shown. In FIG. 2B, a simulation of the fluid drag profile surrounding the cylindrical device subject to a 1 cm/second movement speed is performed to determine drag forces (F_{D}) for diameters ranging from 250 μm to 1750 μm (at every 250 μm). From these simulations, a net propulsion force, Fₙₑₜ, of the cylindrical channel for each diameter was determined using the following equation,

Fₙₑₜ=ρ_{fluid}A_{channel}(V_{fluid}−V_{Device})²−F_{D}

where ρ_{fluid}, A_{channel}, V_{fluid}, and V_{device} are the fluid's density, the cross-sectional area of the channel, and the velocities of the fluid and the device, respectively. As shown in FIG. 2C, the optimal diameters for the cylindrical channel range between 1000 μm to 1500 μm for 300 μm long channels.

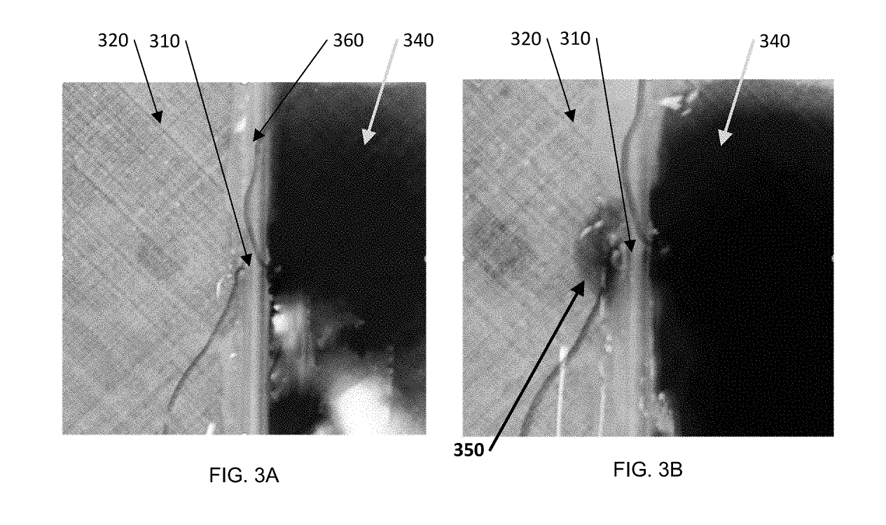

Turning to FIGS. 3A and 3B, to further validate electro-osmotic flow as a propulsion mechanism, an experiment was conducted in which a double-sided device 310 having a cylindrical channel and a gold (Au) surface on each side, was used to separate a left reservoir 320 and a right reservoir 340, each having 25 ml of saline solution. The two reservoirs 320, 340 were further separated by a 1.8 mm thick wall 360 embedded with the double-sided device 310 and, additionally, 200 μL of Propylene Glycol FD&C Blue (blue ink) was deposited into the right reservoir to visualize propulsion of fluid through the cylindrical channel. FIG. 3A shows no movement of the blue ink from the right reservoir 340 to the left reservoir 320 after 9 minutes. In FIG. 3B, an electrical current is applied to the double-sided device 310, and after two minutes, an ink formation 350 is observed in the left reservoir 320. A flow rate through the cylindrical channel was measured as 0.460 μL/sec with an average fluid velocity of 0.586 cm/sec.

### Example Embodiments of Devices for Electro-Osmotic Propulsion

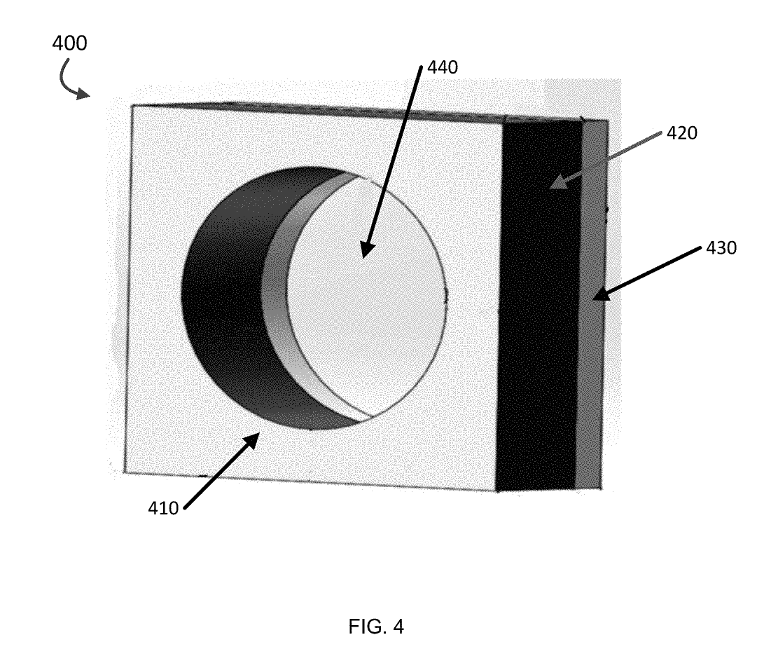

Turning to FIG. 4, a perspective view is provided for an example embodiment of a device 400 for electro-osmotic propulsion in a microfluidic environment. Device 400 includes a body 420 comprising a channel 440 having a pair of open ends. Device 400 also includes a plurality of electrodes 410, 430 coupled to body 420, wherein electrodes 410, 430 are configured to generate a voltage across channel 440 and to cause an electro-osmotic flow of a fluid through channel 440. In some embodiments, a first electrode 410 can be disposed on a first side of body 420, and a second electrode 430 can be disposed on a second side of body 420, wherein the first side is opposite to the second side. In addition, as shown in FIG. 4, channel 440 can be located between the first side and the second side.

In some embodiments, device 400 can be fabricated using a single mask process. For example, body 420 can be fabricated from a p-doped silicon wafer having a particular thickness (e.g., 300 μm). In other embodiments, body 420 can be fabricated from a bio-dissolvable material. Those of skill in the art, however, will recognize that other known methods of manufacture, as well as materials with varying thicknesses can be utilized, depending on the desired voltage, velocity of the device and/or the device application. Additionally, in some embodiments, a thin layer of hydrophobic polymer coating can be applied to some portion of device 400 (e.g., to body 420) to maintain device 400 in a particular orientation.

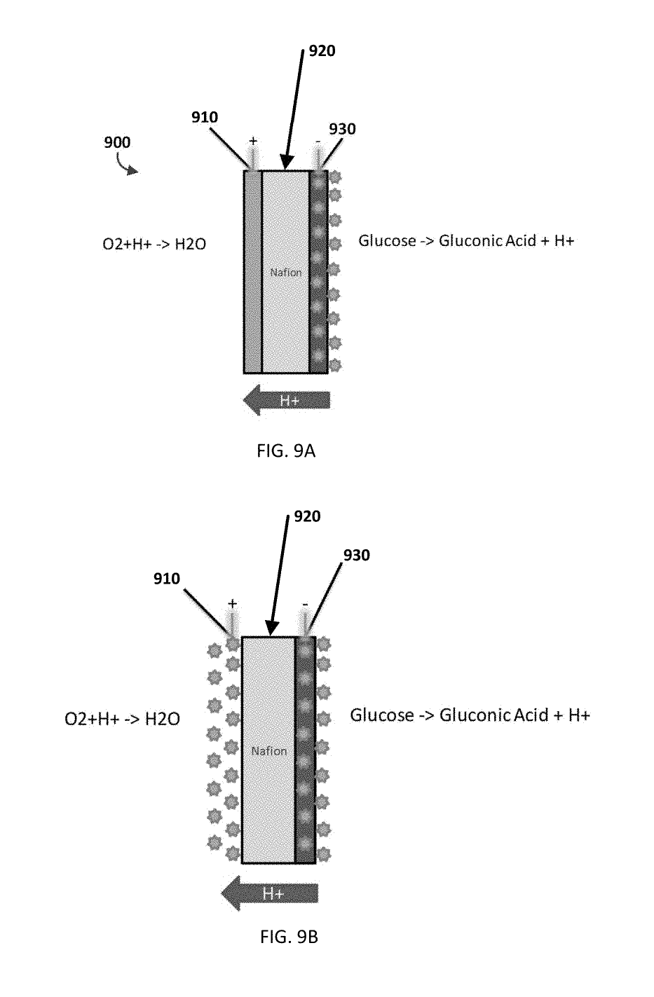

According to one aspect of the disclosed embodiments, device 400 can also include an on-board power supply (not shown) coupled to body 420 of device 400. For example, in some embodiments, the on-board power supply can be a biofuel cell, as described below with respect to FIG. 9. In other embodiments, the on-board power supply can be a redox Aluminum-air battery. For example, with reference to FIG. 4, electrode 430 can be an Al anode, which can be fabricated from an Al foil having a particular thickness (e.g., 100 μm). Similarly, electrode 410 can be an Au cathode which can be fabricated by depositing a layer of Au having a particular thickness (e.g., 200 nm) on the other side of body 420 by electron-beam evaporation. In still other embodiments, the on-board power supply can be a nanopore battery array. In yet other embodiments, the on-board power supply can be a capacitor comprising materials having a high dielectric permittivity. Those of skill in the art will appreciate that these on-board power supplies are intended to be illustrative, and that other types of miniaturized batteries, including other types of electrochemical storage devices, are fully within the scope of the present disclosure.

According to another aspect of the disclosed embodiments, device 400 can receive power from an external power supply (not shown) in addition to, or as an alternative to, the on-board power supply. The external power supply can be located outside of the microfluidic environment, and configured to provide power to device 400 through microwave and/or RF radiation, for example. Those of skill in the art will recognize that other methods of transmitting power to a remote device are possible and are within the scope of the present disclosure.

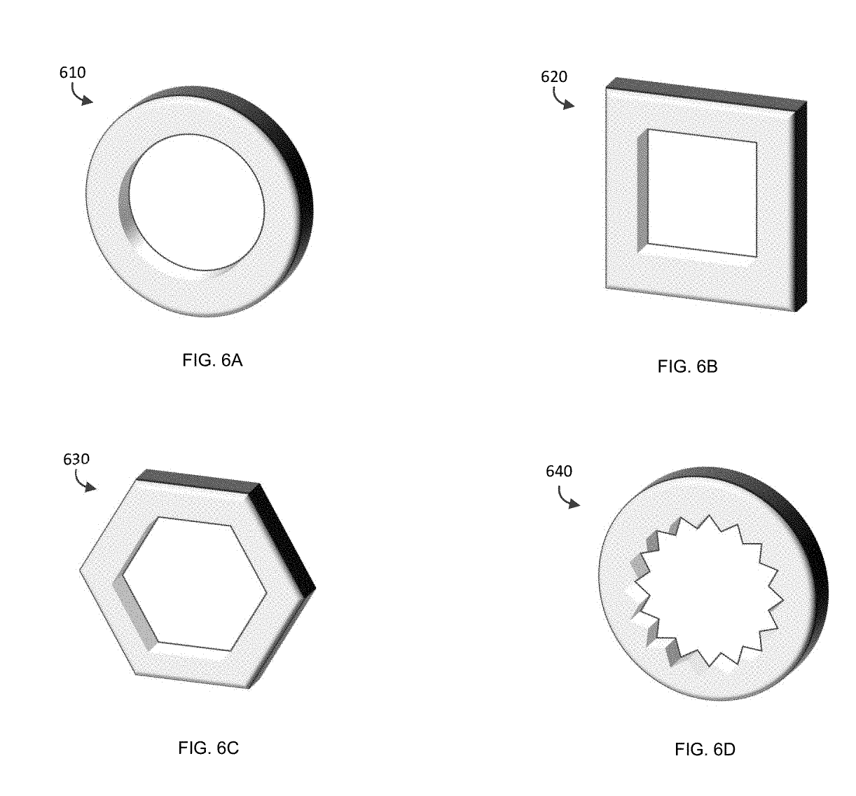

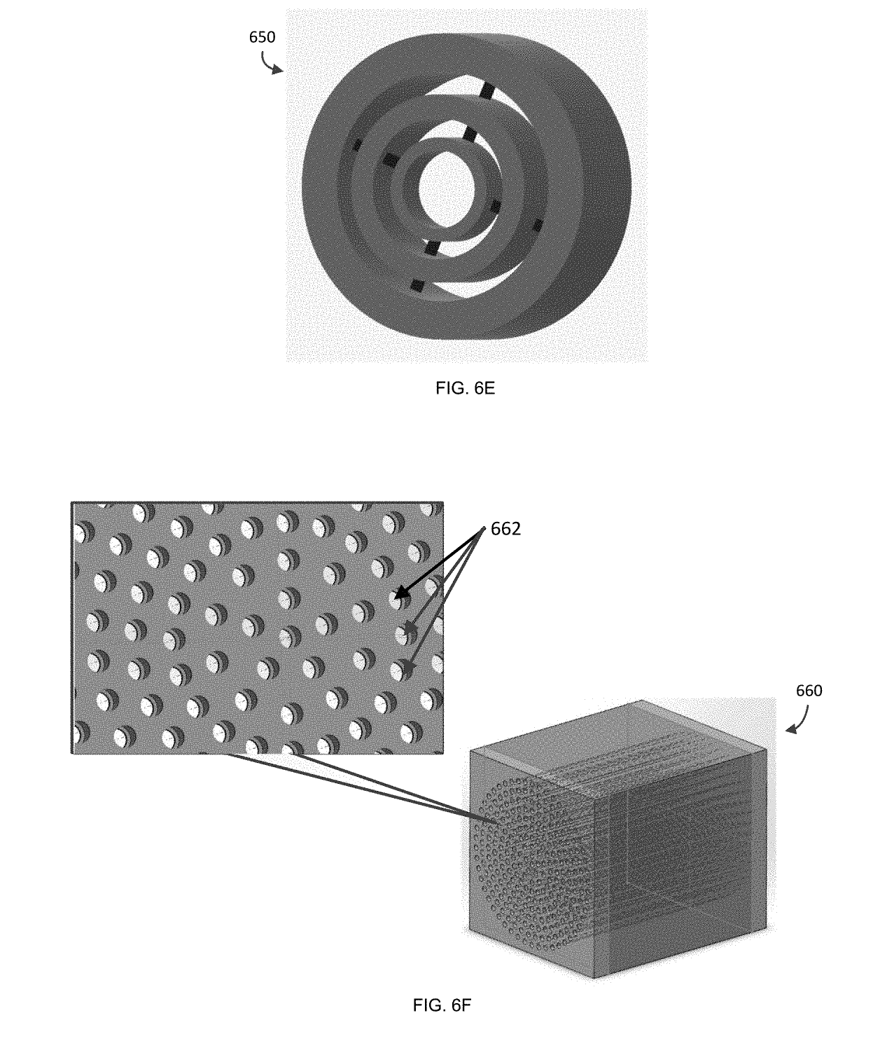

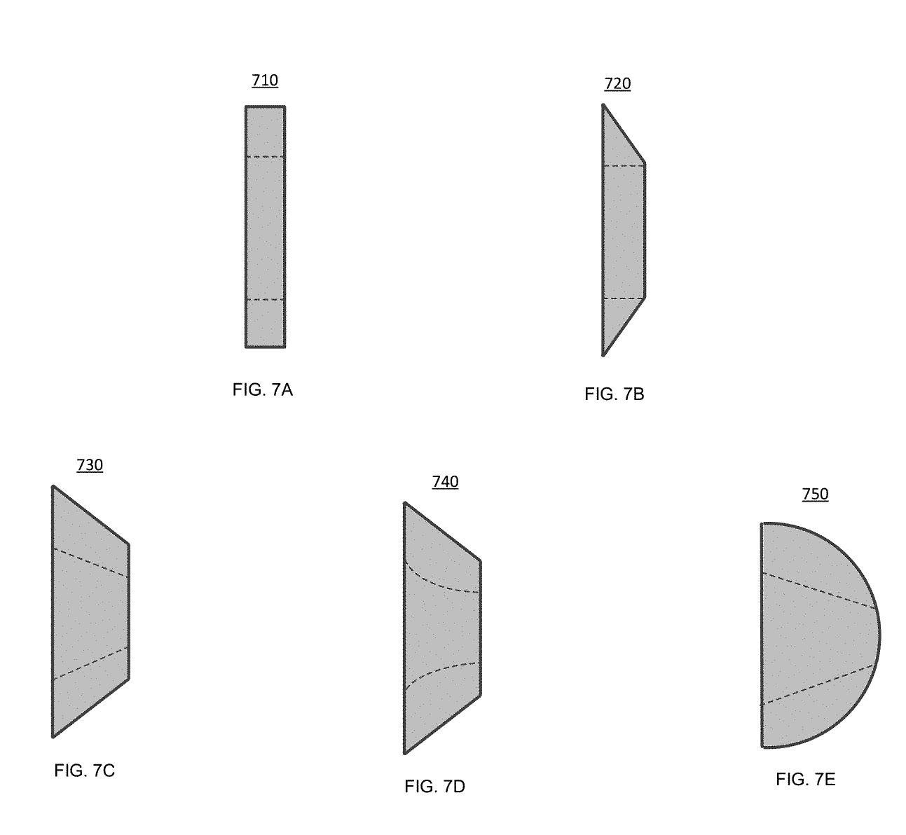

Referring still to FIG. 4, device 400 can have a rectangular outer surface with a cylindrical channel 440 disposed within, wherein cylindrical channel 440 has a particular length (e.g., 300 μm). In some embodiments, cylindrical channel 440 can have a circular cross-sectional area with a particular diameter. For example, the diameter of the circular cross-sectional area can range between 100 nm and 2000 μm. Similarly, the length of the cylindrical channel can range between 100 nm and 1 mm. These configurations are illustrative and not intended to be limiting in any way, and those of skill in the art will appreciate that other lengths, diameters and geometries for device 400, body 420 and channel 440 are possible, as described below with respect to FIGS. 6 and 7.

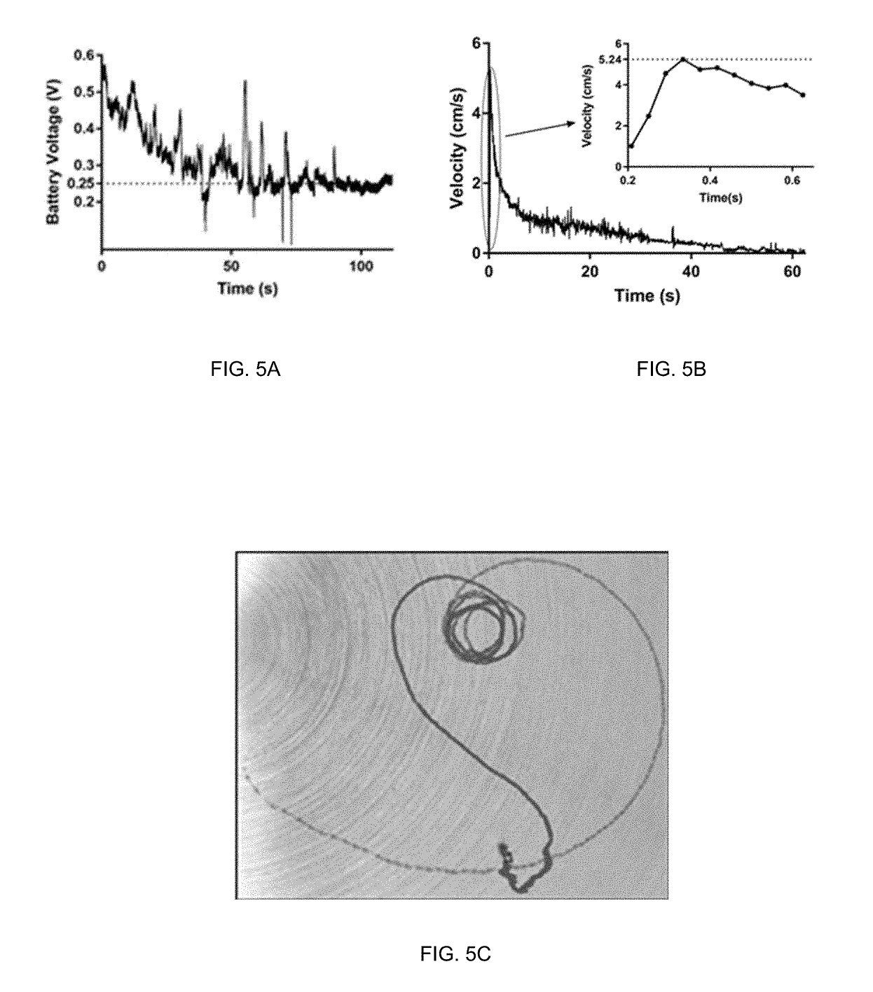

Turning to FIGS. 5A and 5B, provided are graphs showing numerous measurements of battery voltage and velocity, respectively, as a function of time for the example embodiment of a device for electro-osmotic propulsion in a microfluidic environment, as described with respect to FIG. 4. In relation to these figures, experiments were carried out using an example device with a 1000 μm-diameter channel in 7680 μS/cm saline solution, which approximates the median value of blood's conductivity ranging from 2000-15,000 μS/cm, depending on blood cell concentration. The example device included an Al-air battery, and was submerged in the saline solution. The voltage between the Al and Au electrodes was measured at 100 Hz for 120 seconds, and a steady state operating voltage of approximately 250 mV was observed. FIG. 5A depicts the recorded output voltages of the Al-air battery for the example device.

Additionally, video footage of the device's movement was captured. FIG. 5C shows the vectored path of the device movement in the saline solution captured by the video camera. Using video physics analysis software, video footage of the device's movement in the saline solution was analyzed. FIG. 5B depicts a graph of the device's velocity through the video. As can be seen in the inset of FIG. 5B, an initial period of acceleration of up to 5.24 cm/sec was followed by an exponential decay in velocity to a steady state around 1 cm/sec in the first 10 seconds, which was then followed by a gradual decay over the next 52 seconds to a halt. The average velocity for the period was 0.583 cm/sec. The graphs of FIGS. 5A and 5B thus demonstrate a strong correlation between the declining voltage of the Al-air battery and the deceleration of the device. Further, a strong correlation was also demonstrated between the average velocity of the device (0.583 cm/sec) of FIGS. 5A and 5B, and the average fluid velocity of device (0.586 cm/sec) of FIGS. 3A and 3B. Considering the agreement between the velocity curve and the expected pattern from the voltage data and also between the flow rate and the velocity data, the data suggest that electro-osmosis can be an effective propulsion mechanism in microfluidic environments.

Turning to FIGS. 6A to 6F, provided are perspective views of other example embodiments of devices for electro-osmotic propulsion in microfluidic environments. As described above with respect to FIG. 4, an example embodiment of device 400 can have a rectangular outer surface with a cylindrical channel 440 disposed within. In other embodiments, however, different configurations and geometries can be implemented. For example, as shown in FIGS. 6A to 6E, device can include: a circular outer surface with a channel having a circular cross-sectional area (610); a rectangular outer surface with a channel having a rectangular cross-sectional area (620); a hexagonal outer surface with a channel having a hexagonal cross-sectional area (630); a circular outer surface with a channel having a star-shaped cross-sectional area (640); or a circular outer surface with a channel having a cross-sectional area comprising concentric rings (650). In other embodiments, such as those shown in FIG. 6F, device 660 can include multiple parallel channels 662 (or pores), wherein the parallel channels can collectively have a circular cross-sectional area. Those of skill in the art will appreciate that additional combinations of cross-sectional geometries for the outer surfaces of example devices and their respective channels, are fully within the scope of the present disclosure including, but not limited to: triangular, trapezoidal, diamond-shaped, pentagonal, octagonal, decagonal, and other polygonal configurations. Furthermore, for embodiments having a cross-sectional area comprising concentric rings, the number or thickness of the concentric ring walls and the concentric channels therebetween can vary, and are not limited to the depicted embodiment of FIG. 6E. Similarly, for embodiments including multiple parallel channels (or pores), the number, density and cross-sectional configuration of parallel channels can also vary, and are not limited to the embodiment depicted in FIG. 6F.

Turning to FIGS. 7A to 7E, provided are side profile views of other example embodiments of devices. As described above with respect to FIG. 4, an example embodiment of device 400 can have a side profile as shown in FIG. 7A, wherein the dotted lines represent the boundaries of the channel disposed within the device. In other embodiments, however, different configurations and geometries can be implemented. For example, as shown in FIGS. 7B to 7E, device can include: a tapered outer surface (720); a tapered outer surface and a tapered channel (730); a tapered outer surface and curved tapered channel (740); or a curved outer surface and tapered channel (750). Those of skill in the art will appreciate that additional combinations of length-wise geometries for the outer surfaces of example devices and their respective channels, are fully within the scope of the present disclosure and can include, for example, conical, pyramidal, spherical, hemispherical, bell-shaped curves, convex curves, concave curves, textured surfaces and other configurations.

The aforementioned examples are meant to be illustrative and non-limiting, as those of skill in the art will recognize that other geometries for a device having a channel disposed within to permit fluid flow are possible.

### Example Embodiment of Microbot Devices

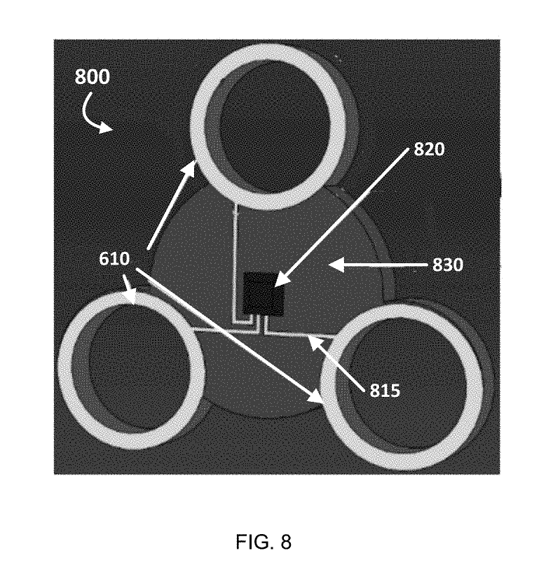

FIG. 8 is a perspective view of an example embodiment of a microbot 800 comprising multiple electro-osmotic propulsion devices 610 for transporting structures and payloads in a microfluidic environment. Microbot 800 can include a plurality of electro-osmotic propulsion devices 610, such as those described with respect to FIGS. 4, 6A to 6F, and 7A to 7D; an on-board power supply 830, such as the example biofuel cell described with respect to FIG. 9; memory (not shown) such as non-transitory memory, RAM, Flash or other types of memory; and an integrated control circuit (IC) 820 coupled to the on-board power supply 830. In many embodiments, microbot 800 can also include an antenna (not shown) configured to wirelessly receive transmissions according to a standard wireless protocol such as 802.11, Bluetooth, Bluetooth Low Energy, NFC, IrDA, UHF or other standard wireless protocols. In some embodiments, the antenna can be a microstrip patch antenna, for example, that feeds into the IC 820. In other embodiments, the antenna can be integrated on IC 820. The antenna can be configured to receive encoded instructions from an external controller (not shown) to actuate electro-osmotic propulsion devices 610. For example, in some embodiments, a user can input direction and speed parameters into an external controller, which will then be encoded into binary format and transmitted via an Rf antenna of the external controller to the microstrip antenna of microbot 800. IC 820 can then process the received signal into a digital format and adjust power received from the on-board power supply accordingly for each electro-osmosis propulsion device 610.

According to another aspect of the disclosed embodiments, IC 820 can comprise one more processors which can include, for example, one or more of a general-purpose central processing unit (“CPU”), a graphics processing unit (“GPU”), an application-specific integrated circuit (“ASIC”), a field programmable gate array (“FPGA”), an Application-specific Standard Products (“ASSPs”), Systems-on-a-Chip (“SOCs”), Programmable Logic Devices (“PLDs”), or other similar components. In some embodiments, IC 820 can comprise one or more processors, microprocessors, controllers, and/or microcontrollers, or a combination thereof, wherein each component can be a discrete chip or distributed amongst (and a portion of) a number of different chips, and collectively, can have the majority of the processing capability.

According to another aspect of the disclosed embodiments, the electro-osmotic propulsion devices 610 can operate in concert to move the microbot 800 in a microfluidic environment such as a patient's circulatory system, and using IC 820, can be controlled for full three-axis movement. Although FIG. 8 depicts an example embodiment of a microbot 800 including three electro-osmotic propulsion devices 610, other embodiments may have one, two, four, five or more electro-osmotic propulsion devices 610 and are fully within the scope of the present disclosure. Similarly, although FIG. 8 depicts microbot 800 as having a relatively flat profile, microbot 800 can also have an elongated, narrow and smooth profile to reduce drag on the microbot 800 as it moves through the microfluidic environment.

In some embodiments, microbot 800 can also include one or more sensors (not shown) coupled to IC 820. The sensors can be configured to detect changes in the microbot's position, orientation, speed, acceleration, temperature, amongst other parameters, and can include, for example, accelerometers, magnetometers, manometers, gyroscope sensors, force sensors, pressure sensors and other similar sensors. Those of skill in the art will understand that the aforementioned types of sensors are not meant to be limiting in any way, and other types of sensors are fully within the scope of the present disclosure.

Additionally, in some embodiments, microbot 800 can also include various sensors and/or microelectromechanical systems (MEMS), depending on the particular application of microbot 800. For example, in some embodiments, microbot 800 can be adapted for targeted drug delivery and can include, within the same housing, a reservoir to store a drug and/or medication, and a pump configured to expel the drug at the target site. The pump can be configured to utilize an electro-osmotic propulsion device 610 coupled to the reservoir to control the amount of drug delivered to the target site. The electro-osmotic propulsion device can be a dedicated pump, or in some embodiments, can serve as a part-time or back-up propulsion device for locomotion of microbot 800. Additionally, in certain embodiments, microbot 800, including electro-osmotic propulsion device 610, can be fabricated from a bio-dissolvable material, such that microbot 800 can be injected into a patient's body to transport a load to a desired location and then dissolve. In other embodiments, microbot 800 can also include an analyte sensor that is adapted to sense an analyte in a bodily fluid, such as, e.g., blood glucose. Microbot 800 can be further configured to store the analyte readings in memory, processed by IC 820 and/or transmitted to the external controller via the antenna.

### Example Embodiments of Biofuel Cells

FIGS. 9A and 9B are block diagrams depicting an example embodiment of a glucose biofuel cell 900, at different stages, for use with devices for electro-osmotic propulsion in a microfluidic environment. Referring to FIG. 9A, biofuel cell 900 can include a first portion 910 comprising a platinum (Pt) electrode, a second portion 930 comprising a plurality of MWCNTs and platinum (Pt) nanoclusters, and a third portion 920 located between the first portion and the second portion, the third portion 920 comprising a proton-permeable nafion membrane. The MWCNTs and Pt of the second portion 930 acts as a reaction site to catalyze the oxidation of glucose and other reactants, thereby producing hydrogen ions (H+). The H+ ions move through third portion 920, i.e., the proton permeable nafion membrane, to the first portion 910 where dissolved O₂ is reduced with the H+ ions into H₂O. This creates a potential difference between the anode and cathode, which is then used to power a load. The benefit of the biofuel cell is that it allows for near-continuous use while a battery would eventually deplete its fuel. The biofuel cell also has an additional advantage in that it allows the microbot 800 to be smaller and lighter, as it need not carry its own fuel.

Throughout this disclosure, the preferred embodiment and examples illustrated should be considered as exemplars, rather than as limitations on the present subject matter, which includes many different embodiments. As used herein, the term “present subject matter,” “system,” “device,” “apparatus,” “method,” “present system,” “present device,” “present apparatus” or “present method” refers to any and all of the embodiments described herein, and any equivalents.

It should also be noted that all features, elements, components, functions, and steps described with respect to any embodiment provided herein are intended to be freely combinable and substitutable with those from any other embodiment. If a certain feature, element, component, function, or step is described with respect to only one embodiment, then it should be understood that that feature, element, component, function, or step can be used with every other embodiment described herein unless explicitly stated otherwise. This paragraph therefore serves as antecedent basis and written support for the introduction of claims, at any time, that combine features, elements, components, functions, and steps from different embodiments, or that substitute features, elements, components, functions, and steps from one embodiment with those of another, even if the following description does not explicitly state, in a particular instance, that such combinations or substitutions are possible. It is explicitly acknowledged that express recitation of every possible combination and substitution is overly burdensome, especially given that the permissibility of each and every such combination and substitution will be readily recognized by those of ordinary skill in the art.

When an element or feature is referred to as being “on” or “adjacent” to another element or feature, it can be directly on or adjacent the other element or feature or intervening elements or features may also be present. In contrast, when an element is referred to as being “directly on” or extending “directly onto” another element, there are no intervening elements present. Additionally, when an element is referred to as being “connected” or “coupled” to another element, it can be directly connected or coupled to the other element or intervening elements may be present. In contrast, when an element is referred to as being “directly connected” or “directly coupled” to another element, there are no intervening elements present.

Furthermore, relative terms such as “inner,” “outer,” “upper,” “top,” “above,” “lower,” “bottom,” “beneath,” “below,” and similar terms, may be used herein to describe a relationship of one element to another. Terms such as “higher,” “lower,” “wider,” “narrower,” and similar terms, may be used herein to describe angular relationships. It is understood that these terms are intended to encompass different orientations of the elements or system in addition to the orientation depicted in the figures.

Although the terms first, second, third, etc., may be used herein to describe various elements, components, regions, and/or sections, these elements, components, regions, and/or sections should not be limited by these terms. These terms are only used to distinguish one element, component, region, or section from another. Thus, unless expressly stated otherwise, a first element, component, region, or section discussed below could be termed a second element, component, region, or section without departing from the teachings of the present subject matter. As used herein, the term “and/or” includes any and all combinations of one or more of the associated list items.

The terminology used herein is for the purpose of describing particular embodiments only and is not intended to be limiting. As used herein, the singular forms “a,” “an,” and “the” are intended to include the plural forms as well, unless the context clearly indicates otherwise. For example, when the present specification refers to “an” assembly, it is understood that this language encompasses a single assembly or a plurality or array of assemblies. It will be further understood that the terms “comprises,” “comprising,” “includes,” and/or “including” when used herein, specify the presence of stated features, integers, steps, operations, elements, and/or components, but do not preclude the presence or addition of one or more other features, integers, steps, operations, elements, components, and/or groups thereof.

Embodiments are described herein with reference to view illustrations that are schematic illustrations. As such, the actual thickness of elements can be different, and variations from the shapes of the illustrations as a result, for example, of manufacturing techniques and/or tolerances are expected. Thus, the elements illustrated in the figures are schematic in nature and their shapes are not intended to illustrate the precise shape of a region and are not intended to limit the scope of the present subject matter.

The foregoing is intended to cover all modifications, equivalents and alternative constructions falling within the spirit and scope of the present subject matter as expressed in the appended claims, wherein no portion of the disclosure is intended, expressly or implicitly, to be dedicated to the public domain if not set forth in the claims. Furthermore, any features, functions, steps, or elements of the embodiments may be recited in or added to the claims, as well as negative limitations that define the present subject matter scope of the claims by features, functions, steps, or elements that are not within that scope.

1. A method of propelling a structure in a microfluidic environment, the method comprising:
generating a voltage across a plurality of electrodes coupled to the structure, wherein the structure includes a channel having a pair of open ends; and
creating an electro-osmotic flow of a fluid, based on the generated voltage, through the channel.

2. The method of claim 1, wherein the voltage is generated by an on-board power supply coupled to the structure.

3. The method of claim 2, wherein the on-board power supply comprises a biofuel cell.

4. The method of claim 2, wherein the on-board power supply comprises an aluminum-air battery.

5. The method of claim 1, wherein the plurality of electrodes includes a gold cathode and an aluminum anode.

6. The method of claim 1, wherein the voltage is generated by an external power supply outside of the microfluidic environment.

7. The method of claim 6, wherein the external power supply is configured to emit microwaves or RF radiation.

8. The method of claim 1, wherein the channel comprises a cylindrical shape.

9. The method of claim 1, wherein the channel comprises a tapered cone shape.

10. The method of claim 1, wherein the channel includes a cross-sectional area having a circular shape.

11. The method of claim 10, wherein the circular cross-sectional area includes a diameter between 100 nm and 2000 μm.

12. The method of claim 11, wherein the channel includes a length between 100 nm and 1 mm.

13. The method of claim 1, wherein the channel includes a cross-sectional area comprising concentric rings.

14. The method of claim 1, wherein the channel includes a cross-sectional area having a rectangular shape.

15. The method of claim 1, wherein the channel includes a cross-sectional area having a polygonal shape.

16. The method of claim 1, wherein the plurality of electrodes comprises a first electrode disposed on a first side of the structure and a second electrode disposed on a second side of the structure, and wherein the first side is opposite to the second side.

17. The method of claim 16, wherein the channel is located between the first side and the second side.

18. The method of claim 1, wherein the structure includes a body comprising a silicon material.

19. The method of claim 1, wherein the structure includes a body comprising a bio-dissolvable material.

20. The method of claim 3, wherein the biofuel cell includes a first portion comprising a platinum electrode, a second portion comprising a plurality of multiwall carbon nanotubes (MWCNTs) and platinum nanoclusters, and a third portion located between the first portion and the second portion, the third portion comprising a proton-permeable nafion membrane.

21. The method of claim 20, further comprising catalyzing, at the second portion, the oxidation of glucose.

22. The method of claim 21, further comprising reducing oxygen, by the hydrogen ions, at the first portion to form water, and creating a potential difference between the first portion and the second portion.

23-62. (canceled)
